(19) United States
(12) Patent Application Publication
(10) Pub. No.: US 2013/0273093 A1
(43) Pub. Date: Oct. 17, 2013

(54) Method for Immunising a Subject against Mycobacterium Tuberculosis or Mycobacterium Bovis

(71) Applicants: Peter Charles Leonard Beverley, Headington (GB); Elma Zaven Tchilian, Headington (GB)

(72) Inventors: Peter Charles Leonard Beverley, Headington (GB); Elma Zaven Tchilian, Headington (GB)

(21) Appl. No.: 13/879,983

(22) PCT Filed: Oct. 17, 2011
(86) PCT No.: PCT/GB2011/052005

§ 371 Date: Jun. 25, 2013

(30) Foreign Application Priority Data
Oct. 18, 2010 (GB) 1017574.3
Mar. 8, 2011 (GB) 1103952.6

(51) Int. Cl. A61K 39/04

(52) U.S. Cl. CPC A61K 39/04
USPC 424/190.1; 424/248.1

(57) ABSTRACT

The invention relates to a method for immunising a subject against Mycobacterium tuberculosis ox Mycobacterium bovis. The method comprises parenterally administering to the subject an immunologically effective amount of a first antigen from the Mycobacterium or a first polynucleotide encoding the first antigen and administering to the lung of the subject an immunologically effective amount of a second antigen from the Mycobacterium or a second polynucleotide encoding the second antigen. The two administration steps are carried out less than four weeks apart. The invention also relates to a kit for carrying out the method of the invention.

### FIELD OF THE INVENTION

The invention relates to a method for immunising a subject against Mycobacterium tuberculosis or Mycobacterium bovis. The method comprises parenterally administering to the subject an immunologically effective amount of a first antigen from the Mycobacterium or a first polynucleotide encoding the first antigen and administering to the lung of the subject an immunologically effective amount of a second antigen from the Mycobacterium or a second polynucleotide encoding the second antigen. The two administration steps are carried out less than four weeks apart. The invention also relates to a kit for carrying out the method of the invention.

### BACKGROUND TO THE INVENTION

Pulmonary tuberculosis (TB) is largely an infection localized to the lung. In humans, it is caused by Mycobacterium tuberculosis (Mtb). In cows, it is caused by a related mycobacterium, Mycobacterium bovis (Mb).

Bacillus Calmette-Guérin (BCG) is the only licensed human vaccine against tuberculosis. BCG is an attenuated version of Mb. It provides partial protection against disseminated TB in infants, but the extent of protection against later pulmonary disease is extremely variable throughout the world (Colditz et al. 1994. JAMA. 271:698-702; and Colditz et al. 1995. Pediatrics. 96:29-35). Unfortunately, BCG is also less protective in much of the developing world where an effective vaccine is most needed. Nevertheless, since BCG is widely used and provides partial protection in infants, it is unethical to abandon its use. A favoured strategy is therefore to develop novel vaccination strategies that can be used to boost BCG-induced immunity.

A common assumption in vaccinology is that optimal protection against a pathogen is obtained by prime boosting. This involves administering a vaccine at least twice; first as a prime and second and subsequently as a boost. Traditionally, the same vaccine is given multiple times as homologous boosts. More recently, heterologous prime boosting, in which the same antigen is administered in different forms, has become popular and several prime boost regimes have been shown to induce strong systemic immune responses.

Prime boosting is considered to be essential in developing immunity against most diseases, but unfortunately homologous prime boosting with parenterally administered BCG has been shown repeatedly in animal models and humans not to be more effective in protecting against tuberculosis than a single immunisation (Tala-Heikkilä et al. 1998 Am J Respir Crit. Care Med. 157:1324-1327; Rodrigues et al. 2005. Lancet. 366:1290-1295; Karonga Prevention Trial Group. 1996 Lancet. 348:17-24). In humans, therefore, BCG is usually administered only once.

In mice, guinea pigs and monkeys, heterologous prime boosting strategies have been employed in which BCG is used as the prime and new subunit vaccines, such as recombinant viral vectors expressing Mtb antigens or recombinant Mb proteins in adjuvants, are used as the boosts. In these strategies, all the primes and boosts are given parenterally. When these potential booster vaccines are given as a single immunisation, without BCG priming, they generate good immune responses and protective immunity equivalent to that provided by a single immunisation with BCG (Skeiky et al. 2004. J Immunol. 172:7618-7628; Olsen et al. 2004. Infect Immun. 72:6148-6150; and Langermans et al. 2005. Vaccine 23:2740-2750). However, these “parenteral” heterologous prime boosting strategies have failed to provide consistently better protection than single immunisation with BCG (Williams et al. 2005. Tuberculosis (Edinb) 85:29-38; Reed et al. 2009. Proc Natl Acad Sci USA 106:2301-2306; Verreck et al. 2009. Ronan et al PLoS One 4:e5264; Sharpe et al. 2010. Clin Vaccine Immunol 17:1170-1182; and Tchilian et al. 2009. Infect Immun 77:622-631). The reasons for this remain undetermined, but because of this failure new vaccination strategies to improve protection are still urgently required.

In contrast to the disappointing effects of parenteral prime boosting strategies, heterologous prime boosting strategies in which the primes and boosts are given via different routes have yielded improved results. Three recombinant adenoviruses expressing Mtb antigens (Ad85A, Ad85a:TB10.4, Ad35-TBS) generate good protection in mice when given intranasally (Wang et al. 2004. J Immunol. 173:6357-6365; Mu et al. 2009. Mol Ther 17:1093-1100; and Radosevic et al. 2007. Infect Immun. 75:4105-4115). More importantly, when used in mice and guinea pigs as an intranasal boost after BCG priming, Ad85A generates consistently improved protection over BCG (Santosuosso et al. 2006. Infect Immun. 74:4634-4643; Forbes et al. 2008. J Immunol. 181:4955-4964; and Xing et al. 2009. Ronan et al PLoS One 4:e5856). However, the prime (BCG) and boost (Ad85A) are given at least 4 weeks apart, such as 8 weeks apart (Santosuosso et al.), 10 weeks apart (Forbes et al.) and 4 weeks apart (Xing et al.).

The parenteral BCG followed by intranasal Ad85A prime boosting strategy seems to be effective because Ad85A prevents the growth of Mtb in the lungs up to day 8 after infection (early phase), while BCG only inhibits Mtb growth 14 days or more after infection (late phase) (Ronan et al. 2009. PLoS One 4:e8235). It is also known that, for intranasal immunisation to provide effective protection in the early phase of Mtb infection, it is essential that the inoculum reaches the deep lung and induces a lung rather than upper respiratory immune response (Ronan et al. 2010. Vaccine 28:5179-5184).

### SUMMARY OF THE INVENTION

The inventors have surprisingly shown that prime boosting is not necessary to generate effective immunity against tuberculosis. In particular, the inventors have surprisingly shown that effective immunity against tuberculosis can be generated if Mtb or Mb antigens are administered both parenterally and into the lung less than four weeks apart. More specifically, the inventors have surprisingly shown that effective immunity against tuberculosis can be generated if Mtb or Mb antigens are administered both parenterally and into the lung at approximately the same time (about an hour apart). This effect is not based on prime boosting because it occurs even when two different Mtb or Mb antigens which do not cross react are administered.

The invention therefore provides a method for immunising a subject against Mycobacterium tuberculosis or Mycobacterium bovis, the method comprising (a) parenterally administering to the subject an immunologically effective amount of a first antigen from the Mycobacterium or a first polynucleotide encoding the first antigen and (b) administering to the lung of the subject an immunologically effective amount of a second antigen from the Mycobacterium or a second polynucleotide encoding the second antigen, wherein steps (a) and (b) are carried out less than four weeks apart. Steps (a) and (b) are preferably carried less than three weeks apart, less than two weeks apart, less than one week apart, less then 48 hours apart, less than 24 hours apart or simultaneously.

The invention also provides a kit for immunising a subject against Mycobacterium tuberculosis or Mycobacterium bovis, the kit comprising (a) an immunologically effective amount of a first antigen from the Mycobacterium or a first polynucleotide encoding the first antigen which is suitable for parenteral administration to the subject and (b) an immunologically effective amount of a second antigen from the Mycobacterium or a second polynucleotide encoding the second antigen which is suitable for administration to the lung of the subject.

### BRIEF DESCRIPTION OF THE FIGURES

FIG. 1 shows the protective efficacy of simultaneous immunisation. (A) C57Bl/6 mice were immunised once simultaneously with BCG intranasally (i.n.) and subcutaneously (s.c.), (B) BCG alone was given once only s.c., BCG s.c./rec85A i.n. or BCGs.c./rec85A s.c. were also administered simultaneously once only, while rec85A on its own was given 3 times at two weekly intervals with appropriate adjuvants, (C) rec85A and ESAT-6₁₋₂₀ peptide were administered 3 times at two weekly interval with adjuvants. Mice were challenged i.n. with Mtb and 4 weeks later sacrificed for enumeration of lung mycobacterial load. * indicates statistically significant difference compared to naïve and *** indicates statistically significant difference from the groups indicated with lines, using Mann Witney test.

FIG. 2 shows that rec85A i.n. induces immunity able to inhibit early Mtb growth. C57Bl/6 mice were immunised once with BCG or 3 times at two weekly intervals with rec85A with cholera toxin i.n. or rec85A with MPL s.c. The mice were challenged i.n. with Mtb and sacrificed at weekly intervals for enumeration of lung mycobacterial load. * indicates statistically significant difference compared to naïve mice * p<0.05, ** p<0.01 and *** p<0.005 using Mann-Witney test.

FIG. 3 shows Mtb cfu after SIM with BCG and subunit vaccines. a. C57BL/6 mice were immunized once with BCG with or without simultaneous administration of 85A s.c. or 85A i.n. Ten weeks later they were challenged with Mtb i.n. and after a further 5 weeks sacrificed for enumeration of lung Mtb cfu. b C57BL/6 mice were immunized once with BCG s.c. or BCG s.c./E6 i.n. and C, BCG s.c./10.4 i.n. Mtb challenge and enumeration of cfu as for a. Representative data from one of two experiments are shown. ***p<0.001, **p<0.01, * p<0.05, one-way ANOVA with Tukey's post test. Data are means±s.e.m.

FIG. 4 shows Mtb cfu after SIM with subunit vaccines. a. C57BL/6 mice were immunized 3 times at 2 weekly intervals with 85A s.c., or E6 i.n. either alone or in combination. Six weeks after the last immunization mice were challenged with Mtb and sacrificed for lung cfu enumeration 5 weeks later. b Mice were immunized 3 times with 85A s.c. or E6 s.c. separately or in combination or c, the same antigens were administered 3 times i.n. before challenge and d, MPL or CT were administered 3 times s.c. or i.n separately or simultaneously before Mtb challenge and enumeration as in a. Representative data from one of two experiments are shown. ***p<0.001, **p<0.01, * p<0.05, one-way ANOVA with Tukey's post test. Data are means±s.e.m.

FIG. 5 shows Mtb cfu after SIM with BCG. In two identical experiments, C57BL/6 mice were immunized once with BCG s.c. or BCG i.n. or simultaneously with the same dose of BCG divided between the s.c./i.n. routes. Ten weeks later they were challenged with Mtb and lung cfu enumerated 5 weeks later. Data from two experiments are shown. ***p<0.001, **p<0.01, * p<0.05, one-way ANOVA with Tukey's post test. Data are means±s.e.m.

FIG. 6 shows the cytokine responses of lung and spleen T cells to antigen 85A. Mice were immunized with BCG s.c. BCG s.c./85A s.c. or BCG s.c./85A i.n. Cells were isolated 10 weeks after immunization and stimulated with pooled 85A peptides for 6 hours. The number of IFNγ, IL-2 and TNF producing cells was determined by flow cytometry of lung and spleen CD4 gated cells (numbers of CD8 cells were too low for reliable analysis). Results are expressed as the means±s.e.m. of 3 or 4 mice per group and are representative of 2 independent experiments. ***p<0.001, **p<0.01, * p<0.05, one-way ANOVA with Tukey's post test. Significant differences only between numbers of spleen IL-2 producing cells. All other groups differ significantly from Naïve group but these comparisons are omitted for simplicity.

FIG. 7 shows the cytokine responses of lung and spleen T cells to antigen 85A or E6. Mice were immunized 3 times at 2 weekly intervals with 85A s.c., 85A i.n., E6 s.c. or E6 i.n. either alone or in combination. Cells were isolated 6 weeks after immunization and stimulated with pooled 85A peptides or E6 for 6 hours. The number of IFNγ, IL-2 and TNF producing cells was determined by flow cytometry of lung and spleen CD4 gated cells. Results are expressed as the means±s.e.m. of 3 or 4 mice per group and are representative of 2 independent experiments. Top three panels show significant or no differences between all cytokines in each group. Bottom panel significant differences between numbers of IFNγ producing cells only. ***p<0.001, **p<0.01, * p<0.05, one-way ANOVA with Tukey's post test. All other groups differ significantly from Naïve group but these comparisons are omitted for simplicity.

FIG. 8 shows the cytokine responses of lung T cells to PPD. Mice were immunized with BCG s.c., BCG i.n. or BCG s.c./i.n. Lung cells were isolated 10 weeks after immunization and stimulated for 12 hours with PPD. The percentages of IFNγ, IL-2 and TNF producing cells was determined by flow cytometry of lung CD4 gated cells. Results are expressed the means±s.e.m. of 3 or 4 mice per group and are representative of 2 independent experiments. Significance difference in numbers of TNF producing cells only. ***p<0.001, **p<0.01, * p<0.05, one-way ANOVA with Tukey's post test. All other groups differ significantly from Naïve group but these comparisons are omitted for simplicity.

FIG. 9 shows the kinetics of Mtb growth after s.c. and i.n. immunization. C57BL/6 mice were immunized once with BCG s.c. (FIG. 9c) or BCG i.n. (FIG. 9c) or 3 times at two weekly intervals with 85A s.c. (FIG. 9a), 85A i.n. (FIG. 9a), E6 s.c. (FIG. 9b) or E6 i.n (FIG. 9b). Ten weeks after immunization with BCG or 4 weeks after the last immunization with 85A or E6 mice were challenged with Mtb and groups of mice sacrificed 7, 14, 21 and 28 days later for enumeration of lung Mtb cfu. Representative data from one of two experiments are shown. ***p<0.001, **p<0.01, *p<0.05, one-way ANOVA with Tukey's post test. Data are means±s.e.m.

### DESCRIPTION OF THE SEQUENCES

SEQ ID NO: 1 shows the polynucleotide sequence encoding the 85A antigen from Mycobacterium bovis. The polynucleotide encoding the 85A antigen from Mycobacterium tuberculosis is identical to SEQ ID NO: 1 except for a single silent nucleotide substitution at nucleotide 1023.

SEQ ID NO: 2 shows the amino acid sequence of the 85A antigen from Mycobacterium bovis used in the Examples. The amino acid sequence of the 85A antigen from Mycobacterium tuberculosis is identical to SEQ ID NO: 2.

SEQ ID NO: 3 shows the polynucleotide sequence encoding the 6 kDa early secreted antigenic target (ESAT-6) from the Mycobacterium tuberculosis.

SEQ ID NO: 4 shows the amino acid sequence of the 6 kDa early secreted antigenic target (ESAT-6) from the Mycobacterium tuberculosis. This includes the first 20 amino acids (i.e. amino acids 1 to 20) which were used in the Example as a synthetic peptide.

### DETAILED DESCRIPTION OF THE INVENTION

It is to be understood that different applications of the disclosed methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a lung” includes “lungs”, reference to “an antigen” includes two or more such antigens, reference to “a subject” includes two or more such subjects, and the like.

All publications, patents and patent applications cited herein, whether supra or infra, are hereby incorporated by reference in their entirety.

### Methods

The present invention provides methods for immunising a subject against Mycobacterium tuberculosis (Mtb) or Mycobacterium bovis (Mb). The method may involve immunising a subject against both Mtb and Mb. This embodiment involves using first and second antigens that are present in both of Mtb and Mb.

The method comprises two steps, which are referred to herein as steps (a) and (b). Step (a) comprises parenterally administering to the subject an immunologically effective amount of a first antigen from the Mycobacterium (i.e. Mtb and/or Mb) or a first polynucleotide encoding the first antigen. Step (b) comprises administering to the lung of the subject an immunologically effective amount of a second antigen from the Mycobacterium (i.e. Mtb and/or Mb) or a second polynucleotide encoding the second antigen. Steps (a) and (b) are carried out less than four weeks apart.

The method of the invention has several advantages. First, the method generates improved protection compared with parenteral administration of both the first and second antigens. Second and more importantly, the method is more convenient than known prime boosting strategies for immunising against Mtb and/or Mb. As will be clear from the discussion below, the two administration steps can be carried out at the same time or on the same day. This means that effective immunity can be genereated with one visit to an immunising cline. This is of course important in some countries, particularly developing countries, where access to immunising clinics is limited.

The invention also provides:

a first antigen from Mycobacterium tuberculosis or Mycobacterium bovis or a first polynucleotide encoding the first antigen and a second antigen from Mycobacterium tuberculosis or Mycobacterium bovis or a second polynucleotide encoding the second antigen for use in a method for immunising a subject against Mycobacterium tuberculosis or Mycobacterium bovis, wherein the method comprises (a) parenterally administering to the subject an immunologically effective amount of the first antigen or the first polynucleotide and (b) administering to the lung of the subject an immunologically effective amount of the second antigen or the second polynucleotide, wherein steps (a) and (b) are carried out less than four weeks apart;

use of (1) a first antigen from Mycobacterium tuberculosis or Mycobacterium bovis or a first polynucleotide encoding the first antigen and (2) a second antigen from Mycobacterium tuberculosis or Mycobacterium bovis or a second polynucleotide encoding the second antigen in the manufacture of a medicament for immunising a subject against Mycobacterium tuberculosis or Mycobacterium bovis, wherein the immunisng comprises (a) parenterally administering to the subject an immunologically effective amount of the first antigen or the first polynucleotide and (b) administering to the lung of the subject an immunologically effective amount of the second antigen or the second polynucleotide, wherein steps (a) and (b) are carried out less than four weeks apart; and

a product containing (1) a first antigen from Mycobacterium tuberculosis or Mycobacterium bovis or a first polynucleotide encoding the first antigen and (2) a second antigen from Mycobacterium tuberculosis or Mycobacterium bovis or a second polynucleotide encoding the second antigen for simultaneous, separate or sequential use in a method for immunising a subject against Mycobacterium tuberculosis or Mycobacterium bovis, wherein the method comprises (a) parenterally administering to the subject an immunologically effective amount of the first antigen or the first polynucleotide and (b) administering to the lung of the subject an immunologically effective amount of the second antigen or the second polynucleotide, wherein steps (a) and (b) are carried out less than four weeks apart.

### Timing and Order of Steps (a) and (b)

Steps (a) and (b) are carried out less than 4 weeks apart (i.e. less than 28 days apart). Steps (a) and (b) are preferably carried out less than three weeks apart, less than two weeks apart or less than one week apart. Steps (a) and (b) may be carried out less than 28, less than 27, less than 26, less than 25, less than 24, less than 23, less than 22, less than 21, less than 20, less than 19, less than 18, less than 17, less than 16, less than 15, less than 14, less than 13, less than 12, less than 11, less than 10, less than 9, less than 8, less than 7 less than, less than 6, less than 5, less than 4, less than 3, less than 2 or less than 1 day(s) apart. Steps (a) and (b) are preferably are carried out less then 48 hours apart, less than 36 hours apart or less than 24 hours apart. Hence, steps (a) and (b) may be carried out on the same day or during the same visit to an immunising clinic. Steps (a) and (b) may be carried out less than 12 hours apart, such as less than 10 hours apart, less than 6 hours apart or less than 2 hours apart. More preferably, steps (a) and (b) are carried out less than 1 hour apart. Most preferably, steps (a) and (b) are carried out simultaneously (i.e. at the same time).

If steps (a) and (b) are carried out at different times, they may be carried out in any order. Step (a) may be carried out before step (b) or step (b) may be carried out before step (a).

### Step (a)

In step (a), an immunologically effective amount of a first antigen from the Mycobacterium (i.e. Mtb and/or Mb) or a first polynucleotide encoding the first antigen is parenterally administered to the subject.

In step (a), an immunologically effective amount is an amount which induces a systemic mononuclear cell response against the antigen in the subject. An immunologically effective amount will also lead to systemic immunisation. In the context of the invention, systemic immunisation is effective if it induces a systemic mononuclear cell response, preferably a systemic T cell response, to the relevant antigen. Systemic immunisation preferably induces systemic mononuclear cell immunity, more preferably systemic T cell immunity, to the antigen and preferably increases protection against Mtb or Mb.

Generally, monuclear cell responses can be measured by taking the cells from the subject in a blood sample, although other types of samples which contain mononuclear cells can be used. The sample may be added directly to the assay or may be processed first. Typically the processing may comprise standard techniques such as gradient centrifugation to separate the mononuclear cells, with resuspension in any suitable volume. Alternatively, the processing may comprise diluting of the sample, for example with water, buffer or media. The sample may be diluted from 1.5 to 100 fold, for example 2 to 50 or 5 to 10 fold.

The processing may comprise separation of components of the sample. Typically mononuclear cells are separated from the samples. The mononuclear cells will comprise the T cells and antigen presenting cells (APCs). In some instances only T cells, such as only CD4⁺ or CD8⁺ T cells, can be purified from the sample. Mononuclear cells and T cells can be separated from the sample using techniques known in the art.

At least one first antigen or at least one first polynucleotide is administered in step (a). However, any number of first antigens or first polynucleotides may be administered, such as 2, 3, 4, 5, 10, 15, 20 or more. Step (a) may involve administering one or more first antigens and one or more first polynucleotides.

### Form of First Antigen

The first antigen may be in the form of a recombinant protein. The first antigen may be fused to another protein, such as another antigen from the Mtb or Mb, to form a fusion protein. Hence, step (a) may comprise administering a fusion protein comprising the first antigen and one or more other antigens from Mtb or Mb.

The first antigen is preferably administered as part of an attenuated mycobacterium or a recombinant attenuated mycobacterium. In other words, step (a) preferably comprises administering an attenuated mycobacterium or a recombinant attenuated mycobacterium comprising the first antigen. The first antigen is more preferably administered as part of an attenuated version or a recombinant attenuated version of the Mycobacterium tuberculosis or Mycobacterium bovis. The recombinant attenuated version of the Mycobacterium tuberculosis is preferably modified to make it non-pathogenic and retains its immunogenicity.

The attenuated version or recombinant attenuated version of the Mycobacterium bovis is preferably Bacillus Calmette-Guérin (BCG). The sequence of the complete genome of BCG Pasteur 1173P2 may be found at GenBank Accesstion No. AM408590 or NC_{—}008769. The BCG may be live. The recombinant BCG is preferably modified to make it more immunogenic.

Attenuated mycobacteria may be produced by culture of virulent organisms under conditions that select for loss of virulence. BCG was selected in this way by repeated passage of virulent Mb in vitro. Attenuated recombinant Mtb and Mb may be generated by deleting known virulence genes and their immunogenicity may be increased by overexpressing highly antigenic genes (Grode et al. 2005. J Clin Investig. 115:2472-2479).

### Parenteral Administration

In step (a), the first antigen or first polynucleotide is administered parenterally. The first antigen or first polynucleotide can be administered by intradermal, subcutaneous, percutaneous, intramuscular, intravenous, intra-arterial, intraperitoneal, intraarticular, intraosseous or other appropriate administration routes. Where a first antigen is to be administered, it is preferred to administer the antigen to a site in the body where it will have the ability to contact suitable antigen presenting cells, and where it, or they, will have the opportunity to contact mononuclear cells of the subject. The first antigen or first polynucleotide is preferably administered close to a lymph node.

### First Polynucleotide

The first polynucleotide may be provided in the form of an expression cassette which includes control sequences operably linked to the inserted sequence, thus allowing for expression of the first antigen in vivo in the subject. These expression cassettes, in turn, are typically provided within vectors (e.g., plasmids or recombinant viral vectors) which are suitable for use as reagents for polynucleotide immunisation. Adenovirus vectors are particularly preferred. Such an expression cassette may be administered directly to the subject. Alternatively, a vector comprising a polynucleotide may be administered to the subject. Preferably the first polynucleotide is prepared and/or administered using a genetic vector. A suitable vector may be any vector which is capable of carrying a sufficient amount of genetic information, and allowing expression of the encoded antigen.

Expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a protein. Other suitable vectors would be apparent to persons skilled in the art. By way of further example in this regard we refer to Sambrook et al (1989, Molecular Cloning—a laboratory manual; Cold Spring Harbor Press).

Preferably, the polynucleotide in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner A control sequence “operably linked” to a coding sequence is ligated in such a way that expression of the coding sequence is achieved under conditions compatible with the control sequences.

A number of expression systems have been described in the art, each of which typically consists of a vector containing a polynucleotide sequence of interest operably linked to expression control sequences. These control sequences include transcriptional promoter sequences and transcriptional start and termination sequences. The vectors may be for example, plasmid, virus or phage vectors provided with an origin of replication, optionally a promoter for the expression of the said polynucleotide and optionally a regulator of the promoter. A “plasmid” is a vector in the form of an extrachromosomal genetic element. The vectors may contain one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example to allow in vivo expression of the antigen.

A “promoter” is a nucleotide sequence which initiates and regulates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

A polynucleotide, expression cassette or vector may additionally comprise a signal peptide sequence. The signal peptide sequence is generally inserted in operable linkage with the promoter such that the signal peptide is expressed and facilitates secretion of a polypeptide encoded by coding sequence also in operable linkage with the promoter.

Typically a signal peptide sequence encodes a peptide of 10 to 30 amino acids for example 15 to 20 amino acids. Often the amino acids are predominantly hydrophobic. In a typical situation, a signal peptide targets a growing polypeptide chain bearing the signal peptide to the endoplasmic reticulum of the expressing cell. The signal peptide is cleaved off in the endoplasmic reticulum, allowing for secretion of the polypeptide via the Golgi apparatus. Thus, a protein may be provided to an individual by expression from cells within the individual, and secretion from those cells.

Alternatively, polynucleotides may be expressed in a suitable manner to allow presentation of the encoded protein by an MHC class II molecule at the surface of an antigen presenting cell. For example, a polynucleotide, expression cassette or vector may be targeted to antigen presenting cells, or the expression of encoded protein may be preferentially stimulated or induced in such cells.

In some embodiments, the polynucleotide, expression cassette or vector will encode an adjuvant, or an adjuvant will otherwise be provided. As used herein, the term “adjuvant” refers to any material or composition capable of specifically or non-specifically altering, enhancing, directing, redirecting, potentiating or initiating an antigen-specific immune response.

Methods for gene delivery are known in the art. See, e.g., U.S. Pat. Nos. 5,399,346, 5,580,859 and 5,589,466. The polynucleotide can be introduced directly into the recipient subject, such as by standard intramuscular or intradermal injection; transdermal particle delivery; inhalation; topically, or by oral, intranasal or mucosal modes of administration. The molecule alternatively can be introduced ex vivo into cells that have been removed from a subject. For example, a polynucleotide, expression cassette or vector may be introduced into APCs of an individual ex vivo. Cells containing the polynucleotides of interest are re-introduced into the subject such that an immune response can be mounted against the proteins encoded by the polynucleotides.

The proteins, polynucleotides, vectors or APCs may be present in a substantially isolated form. They may be mixed with carriers or diluents (as discussed below) which will not interfere with their intended use and still be regarded as substantially isolated. They may also be in a substantially purified form, in which case they will generally comprise at least 90%, e.g. at least 95%, 98% or 99%, of the proteins, polynucleotides, cells or dry mass of the preparation.

### Formulations and Compositions

The first antigen or first polynucleotide is preferably administered together with one or more pharmaceutically acceptable carriers or diluents and optionally one or more other therapeutic ingredients. The carrier (s) must be ‘acceptable’ in the sense of being compatible with the other ingredients of the formulation and not deleterious to the recipient thereof. Typically, carriers for injection, and the final formulation, are sterile and pyrogen free. Preferably, the carrier or diluent is thioglycerol or thioanisole.

Formulation of a suitable composition can be carried out using standard pharmaceutical formulation chemistries and methodologies all of which are readily available to the reasonably skilled artisan.

For example, the first antigen or first polynucleotide can be combined with one or more pharmaceutically acceptable excipients or vehicles. Auxiliary substances, such as wetting or emulsifying agents, pH buffering substances and the like, may be present in the excipient or vehicle. These excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol, thioglycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

Such compositions may be prepared, packaged, or sold in a form suitable for bolus administration or for continuous administration. Injectable compositions may be prepared, packaged, or sold in unit dosage form, such as in ampoules or in multi-dose containers containing a preservative. Compositions include, but are not limited to, suspensions, solutions, emulsions in oily or aqueous vehicles, pastes, and implantable sustained-release or biodegradable formulations. Such compositions may further comprise one or more additional ingredients including, but not limited to, suspending, stabilizing, or dispersing agents. In one embodiment of a composition for parenteral administration, the active ingredient is provided in dry (for e.g., a powder or granules) form for reconstitution with a suitable vehicle (e.g., sterile pyrogen-free water) prior to parenteral administration of the reconstituted composition. The compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Other parentally-administrable compositions which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Alternatively, the first antigen or first polynucleotide may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyomithine, spermine, spermidine, as well as conjugates of these molecules.

The formulation of the first antigen or first polynucleotide will depend upon factors such as the nature of the substances in the composition and the method of delivery. The first antigen or first polynucleotide can be administered in a variety of dosage forms. They may be administered parenterally, subcutaneously, intravenously, intramuscularly, transdermally, intradermally, intraosseously or by infusion techniques. A physician will be able to determine the required route of administration for each particular individual.

The administered compositions will comprise a suitable concentration of the first antigen or first polynucleotide which is effective without causing adverse reaction. Typically, the concentration of each protein in the composition will be in the range of 0.03 to 200 nmol/ml. More preferably in the range of 0.3 to 200 nmol/ml, 3 to 180 nmol/ml, 10 to 150 nmol/ml, 50 to 200 nmol/ml or 30 to 120 nmol/ml. The composition or formulations should have a purity of greater than 95% or 98% or a purity of at least 99%.

An adjuvant may also be used in combination with the first antigen or first polynucleotide. The adjuvant is preferably administered in an amount which is sufficient to augment the effect of the first antigen or first polynucleotide or vice versa. The adjuvant or other therapeutic agent may be an agent that potentiates the effects of the first antigen or first polynucleotide. For example, the other agent may be an immunomodulatory molecule or an adjuvant which enhances the response to the first antigen or first polynucleotide.

In one embodiment, therefore, the first antigen or first polynucleotide is used in combination with one or more other therapeutic agents. The agents may be administered separately, simultaneously or sequentially. They may be administered in the same or different compositions as the first antigen or first polynucleotide. Accordingly, in a method of the invention, the subject may also be treated with a further therapeutic agent.

A composition may therefore be formulated with the first antigen or first polynucleotide and also one or more other therapeutic molecules. The first antigen or first polynucleotide may alternatively be used simultaneously, sequentially or separately with one or more other therapeutic compositions as part of a combined treatment.

Non-limiting examples of adjuvants include alum, monophosphoryl lipid, oligonucleotides, cholera toxin and Freund's incomplete adjuvant.

### Delivery Regimes

Administration of the first antigen or first polynucleotide may be by any suitable method as described above. Suitable amounts of the antigen may be determined empirically, but typically are in the range given below. A single administration of the first antigen or first polynucleotide may be sufficient to have a beneficial effect for the patient, but it will be appreciated that it may be beneficial if step (a) is carried out more than once, in which case typical administration regimes may be, for example, once or twice a week for 2-4 weeks every 6 months, or once a day for a week every four to six months. As will be appreciated, each antigen or polynucleotide, or combination of antigens and/or polynucleotides may be administered to a patient singly or in combination.

Dosages for administration will depend upon a number of factors including the nature of the composition, the route of administration and the schedule and timing of the administration regime. Suitable doses may be in the order of up to 15 μg, up to 20 μg, up to 25 μg, up to 30 μg, up to 50 μg, up to 100 μg, up to 500 μg or more per administration. Suitable doses may be less than 15 μg, but at least 1 ng, or at least 2 ng, or at least 5 ng, or at least 50 ng, or least 100 ng, or at least 500 ng, or at least 1 μg, or at least 10 μg. For some molecules, the dose used may be higher, for example, up to 1 mg, up to 2 mg, up to 3 mg, up to 4 mg, up to 5 mg or higher. Such doses may be provided in a liquid formulation, at a concentration suitable to allow an appropriate volume for administration by the selected route.

For attenuated mycobacteria, doses are measured as the number of organisms administered. Dosages for administration will depend upon a number of factors including the nature of the composition, the route of administration and the schedule and timing of the administration regime. Suitable doses may be in the order of 10³ to 10⁷ organismsper administration. Such doses may be provided in a liquid formulation, at a concentration suitable to allow an appropriate volume for administration by the selected route.

### Step (b)

In step (b), an immunologically effective amount of a second antigen from the Mycobacterium (i.e. Mtb and/or Mb) or a second polynucleotide encoding the second antigen is administered to the lung of the subject.

In step (b), an immunologically effective amount is an amount which induces a pulmonary mononuclear cell response against the antigen in the subject. An immunologically effective amount will also lead to pulmonary immunisation. Pulmonary immunisation is immunisation of the lungs. In the context of the invention, pulmonary immunisation is effective if it induces a pulmonary mononuclear cell response, preferably a pulmonary T cell response, to the relevant antigen. Pulmonary immunisation preferably induces pulmonary mononuclear cell immunity, more preferably pulmonary T cell immunity, to the antigen and preferably increases protection against Mtb or Mb.

Methods are known in the art for isolating and identifying lung mononuclear cells and thereby measuring pulmonary mononuclear cell responses. The mononuclear cells preferably contain T cells. The T cells may be CD8⁺ or CD4⁺ cells.

Lung mononuclear cells may be obtained from a lung biopsy. Lung mononuclear cells are preferably obtained using Bronchoalveolar lavage (BAL). This involves inserting a bronchoscope into the lungs, using the bronchoscope to deliver fluid into a small part of the lung and then recollecting the fluid for examination. The BAL sample is typically processed prior to being used in the invention, for example by centrifugation, by passage through a membrane that filters out unwanted molecules or cells or by lysis of unwanted cells.

At least one second antigen or at least one second polynucleotide is administered in step (b). However, any number of second antigens or second polynucleotides may be administered, such as 2, 3, 4, 5, 10, 15, 20 or more. Step (b) may involve administering one or more second antigens and one or more second polynucleotides.

### Form of Second Antigen or Second Polynucleotide

The second antigen may be in the form of a recombinant protein. The second antigen may be fused to another protein, such as another antigen from the Mtb or Mb, to form a fusion protein. Hence, step (b) may comprise administering a fusion protein comprising the second antigen and one or more other antigens from Mtb or Mb. The second antigen is preferably administered as part of an attenuated mycobacterium or a recombinant attenuated mycobacterium as discussed above. In other words, step (b) preferably comprises administering an attenuated mycobacterium or a recombinant attenuated mycobacterium comprising the second antigen. Any of the attenuated mycobacteria or recombinant attenuated mycobacteria discussed above may be used.

The second polynucleotide may be provided in the form of an expression cassette which includes control sequences operably linked to the inserted sequence, thus allowing for expression of the second antigen in vivo in the subject as discussed above.

The second antigen can be the same as the first antigen. Alternatively, the second antigen can be different from the first antigen. Preferred antigens and combinations of antigens are discussed in more detail below.

In a preferred embodiment, the first antigen and the second antigen do not cross react. Antigens A and B are cross-reactive if a mononuclear cell, such as a T cell, specific for antigen A also recognises antigen B and vice versa when recognition is the binding of the mononuclear cell receptor to a peptide from the antigen when presented on an MHC molecule. In other words, in a preferred embodiment, a mononuclear cell, such as a T cell, specific for the first antigen does not recognise the second antigen and vice versa. An advantage of these preferred embodiments is that they do not rely on prime boosting to generate effective immunity.

### Lung Administration

In step (b), the second antigen or second polynucleotide is administered to the lung of the subject. The second antigen or second polynucleotide is preferably administered to both lungs of the subject. The second antigen or second polynucleotide is administered to the deep lung(s) of the subject. This allows a pulmonary mononuclear cell response and pulmonary immunity to be generated.

The second antigen or second polynucleotide may administered to the lung of the subject using any method. The second antigen or second polynucleotide is preferably administered by inhalation, intranasally, intrasternally or intraorally (e.g. as dispersible aqueous or oily suspensions, dispersible powders or nanoparticles).

### Formulations and Compositions

The second antigen or second polynucleotide is administered is a form suitable for administration to the lung of the subject. Suitable forms are known in the art and are discussed above with reference to step (a). The second antigen or second polynucleotide is preferably administered together with one or more pharmaceutically acceptable carriers or diluents and optionally one or more other therapeutic ingredients. The carrier (s) must be ‘acceptable’ in the sense of being compatible with the other ingredients of the formulation and not deleterious to the recipient thereof. Suitable formulations for lung delivery of the second antigen or second polynucleotide are known in the art. In particular suitable formulations are disclosed in Lu and Hicky 2007. Expert Rev. Vaccines 6(2), 213-226 and Garcia-Contreras et al. 2008. PNAS 105(12): 4656-4660.

### Delivery Regimes

The second antigen or second polynucleotide may be administered using any regime discussed above with reference to step (a). Dosages for administration will depend upon a number of factors including the nature of the composition, the route of administration and the schedule and timing of the administration regime. Any of the suitable doses discussed above may be used. The dose is sufficiently high to allow the second antigen or second polynucleotide to reach the deep lung and generate a pulmonary mononuclear cell response.

### Mtb or Mb

The Mtb or Mb may be any known strain. Suitable strains and their GenBank accession numbers are summarized in Table 1 below.

TABLE 1
Mtb or Mb

| Mycobacterium | Strain | Accession number |
|---|---|---|
| Mtb | H37Rv | AL123456 |
| Mb | subsp. bovis AF2122/97 | BX248333 |

### Subject

In any of the methods discussed above, the subject may be any mammal that is capable of infection with Mtb and/or Mb. Most preferably, the subject is human. However, it may be non-human Suitable non-human animals include, but are not limited to, primates, such as marmosets or monkeys, commercially farmed animals, such as horses, cows, sheeps, goats, alpacas, guanacos, deer or pigs, pets, such as dogs, cats, mice, rats, guinea pigs, ferrets, gerbils or hamsters or wild animals such as badgers or deer. The subject is preferably human, a mouse, pig, a cow or a sheep. The subject is more preferably a human or a cow.

### Antigens

Any antigens from Mtb and/or Mb may be used as long as they generate protective immune responses. Suitable antigens are known in the art.

### 85 Antigens

The first and/or second antigen is preferably the 85A, 85B or 85C antigen from Mtb or Mb or a variant thereof. The 85A, 85B or 85C antigen or a variant thereof may be a recombinant antigen or be part of an attenuated version of Mtb or Mb. A variant of the 85A, 85B or 85C antigen is a protein that has an amino acid sequence which varies from that of the 85A, 85B or 85C antigen and which retains its ability to induce an immune response and generate immunity against Mtb and/or Mb. The ability of a variant to do this can be assayed using any method known in the art. For instance, it can be done as shown in the Example.

The variant may be a naturally occurring variant or a non-naturally occurring variant produced by recombinant technology. Over the entire length of the amino acid sequence of the 85A, 85B or 85C antigen, a variant will preferably be at least 50% homologous to that sequence based on amino acid identity. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of the 85A, 85B or 85C antigen over the entire sequence.

Standard methods in the art may be used to determine homology. For example the UWGCG Package provides the BESTFIT program which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSP's containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The first and/or second antigen preferably comprises the 85A antigen from Mycobacterium bovis and Mycobacterium tuberculosis shown in SEQ ID NO: 2 or a variant thereof. A variant of SEQ ID NO: 2 is a protein that has an amino acid sequence which varies from that of SEQ ID NO: 2 and which retains its ability to induce a mononuclear cell immune response and generate immunity against Mb or Mtb respectively. The ability of a variant to do this can be assayed using any method known in the art. For instance, it can be done as shown in the Example.

The variant may be a naturally occurring variant or a non-naturally occurring variant produced by recombinant technology. Over the entire length of the amino acid sequence of SEQ ID NO: 2, a variant will preferably be at least 50% homologous to that sequence based on amino acid identity. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 2 over the entire sequence. There may be at least 80%, for example at least 85%, 90% or 95%, amino acid identity over a stretch of 150 or more, for example 200, 250 or 300 or more, contiguous amino acids (“hard homology”). Homology may be deteremined as discussed above.

Amino acid substitutions may be made to the amino acid sequence of SEQ ID NO: 2, for example up to 1, 2, 3, 4, 5, 10, 20, 30, 40 or 50 or more substitutions. Conservative substitutions may be made. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 2 below. Where amino acids have similar polarity, this can also be determined by reference to the hydropathy scale for amino acid side chains in Table 3.

TABLE 2
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, |
| Asp | polar, hydrophilic, charged (−) | | neutral |
| Glu | polar, hydrophilic, charged (−) | Pro | hydrophobic, neutral |
| Phe | aromatic, hydrophobic, neutral | Gln | polar, hydrophilic, |
| Gly | aliphatic, neutral | | neutral |
| His | aromatic, polar, hydrophilic, | Arg | polar, hydrophilic, |
| | charged (+) | | charged (+) |
| Ile | aliphatic, hydrophobic, neutral | Ser | polar, hydrophilic, |
| Lys | polar, hydrophilic, charged(+) | | neutral |
| Leu | aliphatic, hydrophobic, neutral | Thr | polar, hydrophilic, |
| | | | neutral |
| | | Val | aliphatic, hydrophobic, |
| | | | neutral |
| | | Trp | aromatic, hydrophobic, |
| | | | neutral |
| | | Tyr | aromatic, polar, |
| | | | hydrophobic |

TABLE 3
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One or more amino acid residues of the amino acid sequence of SEQ ID NO: 2 may additionally be deleted from the polypeptides described above. Up to 1, 2, 3, 4, 5, 10, 20, 30, 40 or 50 residues may be deleted, or more.

Variants may be fragments of SEQ ID NO: 2. Such fragments retain the ability to induce a mononuclear immune response and immunity to Mtb and/or Mb. Fragments may be at least 50, 100, 200, 250 or 300 amino acids in length. A fragment preferably comprises the domains of SEQ ID NO: 2 which interact with MHC molecules and mononuclear cell receptors.

One or more amino acids may be alternatively or additionally added to the polypeptides described above. An extension may be provided at the amino terminus or carboxy terminus of the amino acid sequence of SEQ ID NO: 2 or a variant or fragment thereof. The extension may be quite short, for example from 1 to 10 amino acids in length. Alternatively, the extension may be longer, for example up to 50 or 100 amino acids. A carrier protein may be fused to SEQ ID NO: 2 or a variant thereof.

The variant may be modified for example by the addition of histidine or aspartic acid residues to assist its identification or purification or by the addition of a signal sequence to promote their secretion from a cell where the polypeptide does not naturally contain such a sequence.

The antigen may be labelled with a revealing label. The revealing label may be any suitable label which allows the protein to be detected. Suitable labels include, but are not limited to, fluorescent molecules, radioisotopes, e.g. ¹²⁵I, ³⁵S, ¹⁴C, enzymes, antibodies, antigens, polynucleotides and ligands such as biotin.

The antigen may be isolated from Mtb or Mb, or made synthetically or by recombinant means. For example, the protein may be synthesised by in vitro translation and transcription. The amino acid sequence of the antigen may be modified to include non-naturally occurring amino acids or to increase the stability of the antigen. When the antigen is produced by synthetic means, such amino acids may be introduced during production. The antigen may also be altered following either synthetic or recombinant production.

The antigen may also be produced using D-amino acids. For instance, the antigen may comprise a mixture of L-amino acids and D-amino acids. This is conventional in the art for producing such antigens.

The antigen may also contain other non-specific chemical modifications as long as they do not interfere with its ability to induce a mononuclear cell response. A number of non-specific side chain modifications are known in the art and may be made to the side chains of the antigen. Such modifications include, for example, reductive alkylation of amino acids by reaction with an aldehyde followed by reduction with NaBH₄, amidination with methylacetimidate or acylation with acetic anhydride.

The antigen can be produced using standard methods known in the art. Polynucleotide sequences encoding the antigen or variant thereof may be isolated and replicated using standard methods in the art. Chromosomal DNA may be extracted from Mtb or Mb. The gene encoding the antigen may be amplified using PCR involving specific primers. The amplified sequence may then be incorporated into a recombinant replicable vector such as a cloning vector. The vector may be used to replicate the polynucleotide in a compatible host cell. Thus polynucleotide sequences encoding the antigen may be made by introducing a polynucleotide encoding the antigen into a replicable vector, introducing the vector into a compatible host cell, and growing the host cell under conditions which bring about replication of the vector. The vector may be recovered from the host cell.

The polynucleotide sequence may be cloned into a suitable expression vector. In an expression vector, the polynucleotide sequence encoding the antigen is typically operably linked to a control sequence which is capable of providing for the expression of the coding sequence by the host cell. Such expression vectors can be used to express the antigen.

“Operably linked” is defined above. Multiple copies of the same or different polynucleotide may be introduced into the vector.

The expression vector may then be introduced into a suitable host cell. Thus, an antigen can be produced by inserting a polynucleotide sequence encoding an antigen into an expression vector, introducing the vector into a compatible bacterial host cell, and growing the host cell under conditions which bring about expression of the polynucleotide sequence.

The vectors may be for example, plasmid, virus or phage vectors provided with an origin of replication, optionally a promoter for the expression of the said polynucleotide sequence and optionally a regulator of the promoter. The vectors may contain one or more selectable marker genes, for example an ampicillin resistance gene. Promoters and other expression regulation signals may be selected to be compatible with the host cell for which the expression vector is designed. A T7, trc, lac, ara or λ_{L} promoter is typically used.

The host cell typically expresses the antigen at a high level. Host cells transformed with a polynucleotide sequence will be chosen to be compatible with the expression vector used to transform the cell. The host cell is typically bacterial and preferably E. coli. Any cell with a λ DE3 lysogen, for example C41 (DE3), BL21 (DE3), JM109 (DE3), B834 (DE3), TUNER, Origami and Origami B, can express a vector comprising the T7 promoter.

An antigen may be produced in large scale following purification by any protein liquid chromatography system from chemokine producing organisms or after recombinant expression as described below. Typical protein liquid chromatography systems include FPLC, AKTA systems, the Bio-Cad system, the Bio-Rad BioLogic system and the Gilson HPLC system.

The first and/or second polynucleotide preferably encodes the 85A, 85B or 85C antigen from Mtb or Mb or a variant thereof. The variant may be a naturally occurring variant or a non-naturally occurring variant produced by recombinant technology. Over the entire length of the sequence of the polynucleotide, a variant will preferably be at least 50% homologous to that sequence based on nucleotide identity. More preferably, the variant may be at least 55%, at least 60%, at least 65%, at least 70%, at least 75%, at least 80%, at least 85%, at least 90% and more preferably at least 95%, 97% or 99% homologous based on nucleotide identity to the polynucleotide over the entire sequence. The polynucleotide may encode any of the antigen variants discussed above.

The first and/or second polynucleotide preferably comprises SEQ ID NO: 1 or a variant thereof A variant of SEQ ID NO: 1 is a polynucleotide that has a sequence which varies from that of SEQ ID NO: 1 and which retains its ability to encode a variant of SEQ ID NO: 2 respectively.

The variant may be a naturally occurring variant or a non-naturally occurring variant produced by recombinant technology. Over the entire length of the sequence of SEQ ID NO: 1, a variant will preferably be at least 50% homologous to that sequence based on nucleotide identity. More preferably, the variant may be at least 55%, at least 60%, at least 65%, at least 70%, at least 75%, at least 80%, at least 85%, at least 90% and more preferably at least 95%, 97% or 99% homologous based on nucleotide identity to the sequence of SEQ ID NO: 1 over the entire sequence. There may be at least 80%, for example at least 85%, 90% or 95%, nucleotide identity over a stretch of 450 or more, for example 600, 750 or 900 or more, contiguous nucleotides (“hard homology”). Homology can be determined as discussed above. The polynucleotide may encode any of the variants of SEQ ID NO: 2 discussed above.

Polynucleotides can be made as discussed above.

### TB10.4 Antigen

The first and/or second antigen may be the antigen TB 10.4 (Dietrich J et al, J Immunol 2005, 174, 6332-6339) or a variant thereof. The TB10.4 antigen or a variant thereof may be a recombinant antigen or be part of an attenuated version of Mtb or Mb. A variant of the TB10.4 antigen is a protein that has an amino acid sequence which varies from that of the TB 10.4 antigen and which retains its ability to induce an immune response and generate immunity against Mtb and/or Mb. The ability of a variant to do this can be assayed using any method known in the art. For instance, it can be done as shown in the Example.

The variant may be a naturally occurring variant or a non-naturally occurring variant produced by recombinant technology. Over the entire length of the amino acid sequence of the TB10.4 antigen, a variant will preferably be at least 50% homologous to that sequence based on amino acid identity. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of the TB10.4 antigen over the entire sequence.

### The Enduring Hypoxia Response Protein Rv1284

The first and/or second antigen may be the enduring hypoxia response protein Rv1284 (Rustad, T. R., Harrell, M. I., Liao, R. & D. R., S. PLoS One. 3, e1502. (2008)) or a variant thereof. The Rv1284 antigen or a variant thereof may be a recombinant antigen or be part of an attenuated version of Mtb or Mb. A variant of the Rv1284 antigen is a protein that has an amino acid sequence which varies from that of the Rv1284 antigen and which retains its ability to induce an immune response and generate immunity against Mtb and/or Mb. The ability of a variant to do this can be assayed using any method known in the art. For instance, it can be done as shown in the Example.

The variant may be a naturally occurring variant or a non-naturally occurring variant produced by recombinant technology. Over the entire length of the amino acid sequence of the Rv1284 antigen, a variant will preferably be at least 50% homologous to that sequence based on amino acid identity. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of the Rv1284 antigen over the entire sequence.

### ESAT6

The first and/or second antigen is preferably the 6 kDa early secreted antigenic target (ESAT-6) from Mtb or Mb or a variant thereof. Other antigens from the RD 1 region of Mtb or Mb which have been deleted in BCG can also be used if they are immunogenic in experimental animals.

A variant of ESAT6 is a protein that has an amino acid sequence which varies from that of ESAT6 and which retains its ability to induce an immune response and generate immunity against Mtb and/or Mb. The ability of a variant to do this can be assayed using any method known in the art. For instance, it can be done as shown in the Example.

The variant may be a naturally occurring variant or a non-naturally occurring variant produced by recombinant technology. A variant of ESAT6 can vary from naturally-occurring ESAT6 in any of the ways discussed above for the 85 antigens.

The first and/or second antigen preferably comprises ESAT6 from Mtb shown in SEQ ID NO: 4 or a variant thereof. A variant of SEQ ID NO: 4 is a protein that has an amino acid sequence which varies from that of SEQ ID NO: 4 and which retains its ability to induce an immune response and generate immunity against Mtb.

The variant may be a naturally occurring variant or a non-naturally occurring variant produced by recombinant technology. Over the entire length of the amino acid sequence of SEQ ID NO: 4, a variant will preferably be at least 50% homologous to that sequence based on amino acid identity. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 4 over the entire sequence. There may be at least 80%, for example at least 85%, 90% or 95%, amino acid identity over a stretch of 20 or more, for example 25, 30, 40, 50 or more, contiguous amino acids (“hard homology”). Homology may be deteremined as discussed above.

Amino acid substitutions may be made to the amino acid sequence of SEQ ID NO: 4, for example up to 1, 2, 3, 4, 5, 10, 15, 20, 25 or 30 or more substitutions. Conservative substitutions may be made as discussed above.

One or more amino acid residues of the amino acid sequence of SEQ ID NO: 4 may additionally be deleted from the polypeptides described above. Up to 1, 2, 3, 4, 5, 10, 15 or 20 residues may be deleted, or more.

Variants may be fragments of SEQ ID NO: 4. Such fragments retain the ability to induce a mononuclear immune response and immunity to Mtb and/or Mb. Fragments may be at least 10 or 15 amino acids in length. A fragment preferably comprises the domains of SEQ ID NO: 4 which interact with MHC molecules and mononuclear cell receptors. The most preferred fragment of SEQ ID NO: 4 is the first 20 amino acids. This fragment is used in the Example.

One or more amino acids may be alternatively or additionally added to the polypeptides described above. An extension may be provided at the amino terminus or carboxy terminus of the amino acid sequence of SEQ ID NO: 4 or a variant or fragment thereof. The extension may be quite short, for example from 1 to 10 amino acids in length. Alternatively, the extension may be longer, for example up to 50 or 100 amino acids. A carrier protein may be fused to SEQ ID NO: 4 or a variant thereof.

The variant may be modified is any of the ways discussed above for the 85 antigens.

The first and/or second polynucleotide preferably encodes the ESAT6 antigen from Mtb or Mb or a variant thereof. The variant may be a naturally occurring variant or a non-naturally occurring variant produced by recombinant technology. The polynucleotide may encode any of the antigen variants discussed above.

The first and/or second polynucleotide preferably comprises SEQ ID NO: 3 or a variant thereof. A variant of SEQ ID NO: 3 is a polynucleotide that has a sequence which varies from that of SEQ ID NO: 3 and which retains its ability to encode a variant of SEQ ID NO: 4.

The variant may be a naturally occurring variant or a non-naturally occurring variant produced by recombinant technology. Over the entire length of the sequence of SEQ ID NO: 3, a variant will preferably be at least 50% homologous to that sequence based on nucleotide identity. More preferably, the variant may be at least 55%, at least 60%, at least 65%, at least 70%, at least 75%, at least 80%, at least 85%, at least 90% and more preferably at least 95%, 97% or 99% homologous based on nucleotide identity to the sequence of SEQ ID NO: 3 over the entire sequence. There may be at least 80%, for example at least 85%, 90% or 95%, nucleotide identity over a stretch of 60 or more, for example 75, 90, 120 or 150 or more, contiguous nucleotides (“hard homology”). Homology can be determined as discussed above. The polynucleotide may encode any of the variants of SEQ ID NO: 4 discussed above.

### Preferred Combinations

Preferred combinations of antigens for use in the invention are shown below in Table 4.

TABLE 4
Preferred combinations of antigens

| First antigen | Second antigen |
|---|---|
| BCG or an attenuated | BCG or an attenuated mycobacterium |
| mycobacterium | |
| BCG or an attenuated | 85A or variant therof |
| mycobacterium | |
| BCG or an attenuated | 85B or variant therof |
| mycobacterium | |
| BCG or an attenuated | 85C or variant therof |
| mycobacterium | |
| BCG or an attenuated | ESAT 6 or variant therof |
| mycobacterium | |
| BCG or an attenuated | SEQ ID NO: 2 or variant thereof |
| mycobacterium | |
| BCG or an attenuated | SEQ ID NO: 4 or variant thereof |
| mycobacterium | |
| 85A or variant therof | BCG or an attenuated mycobacterium |
| 85B or variant therof | BCG or an attenuated mycobacterium |
| 85C or variant therof | BCG or an attenuated mycobacterium |
| ESAT 6 or variant therof | BCG or an attenuated mycobacterium |
| SEQ ID NO: 2 or variant thereof | BCG or an attenuated mycobacterium |
| SEQ ID NO: 4 or variant thereof | BCG or an attenuated mycobacterium |
| 85A or variant therof | 85A or variant therof |
| 85A or variant therof | SEQ ID NO: 2 or variant thereof |
| 85A or variant therof | ESAT 6 or variant therof |
| 85A or variant therof | SEQ ID NO: 4 or variant thereof |
| SEQ ID NO: 2 or variant thereof | 85A antigen or variant therof |
| ESAT 6 antigen or variant therof | 85A antigen or variant therof |
| SEQ ID NO: 4 or variant thereof | 85A antigen or variant therof |
| SEQ ID NO: 2 or variant thereof | SEQ ID NO: 2 or variant thereof |
| SEQ ID NO: 2 or variant thereof | ESAT 6 or variant therof |
| SEQ ID NO: 2 or variant thereof | SEQ ID NO: 4 or variant thereof |
| ESAT 6 or variant therof | SEQ ID NO: 2 or variant thereof |
| SEQ ID NO: 4 or variant thereof | SEQ ID NO: 2 or variant thereof |
| ESAT 6 or variant therof | ESAT 6 or variant therof |
| ESAT 6 or variant therof | SEQ ID NO: 4 or variant thereof |
| SEQ ID NO: 4 or variant thereof | ESAT 6 or variant therof |

### Kit

The invention also provides kits for immunising a subject against Mycobacterium tuberculosis or Mycobacterium bovis. The kits comprise (a) an immunologically effective amount of a first antigen from the Mycobacterium or a polynucleotide encoding the first antigen which is suitable for parenteral administration to the subject and (b) an immunologically effective amount of a second antigen from the Mycobacterium or a second polynucleotide encoding the second antigen which is suitable for administration to the lung of the subject. Any of the antigens discussed above may be used.

The kits of the invention may additionally comprise one or more other reagents or instruments which enable any of the embodiments mentioned above to be carried out. Such reagents or instruments include one or more of the following: suitable buffer(s) (aqueous solutions), means to parenterally administer the first antigen or first polynucleotide to a subject (such as a vessel or an instrument comprising a needle) and means to administer to the lung of the subject the second antigen or second polynucleotide. Reagents may be present in the kit in a dry state such that a fluid sample resuspends the reagents. The kit may also, optionally, comprise instructions to enable the kit to be used in the method of the invention or details regarding which patients the method may be used for.

The following Example illustrates the invention.

### Example 1

### 1.1 Materials and Methods

### 1.1.1 Mice and Immunisation

All experiments were performed with 6-8 week old female C57BL/6 mice (Harlan Orlac, Blackthorn, UK), were approved by the animal use ethical committee of Oxford University and fully complied with the relevant Home Office guidelines. Mice were immunised with 2×10⁵ CFU BCG subcutaneously (s.c.) in 200 μl PBS (BCG SSI, kindly provided by Dr Amy Yang, CBER.FDA, Maryland). For intransal (i.n) immunisation mice were anesthetised with isofluorane and allowed to inhale 40 μl of PBS containing 2×10⁵ CFU BCG. For i.n. and subcutaneous (s.c) immunisation half the dose was administered by each route, 1×10⁵ CFU s.c. and 1×10⁵ cfu i.n, approximately one hour apart.

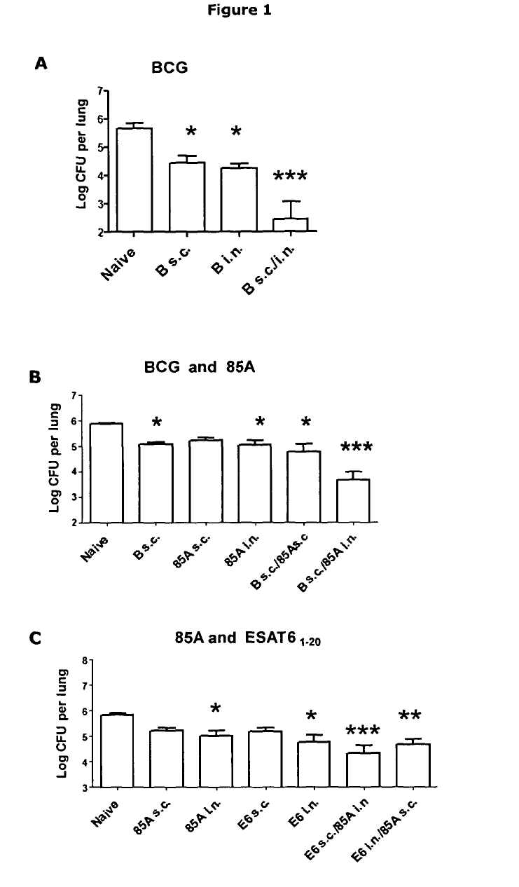

Mice were also immunised with recombinant antigen 85A protein (rec85A) or peptide encoding the first 20 amino acids of ESAT 6 (6 kDa early secreted antigenic target, ESAT6₁₋₂₀). I.n. protein immunisation was conducted as above, delivering 2 μg rec85A protein or 20 μg of ESAT6₁₋₂₀ mixed with 2 μg of cholera toxin (Sigma) into the nostrils. The rec85A or ESAT6₁₋₂₀ were also delivered by the subcutaneous route (s.c.). 2 μg rec85A protein or 20 μg of ESAT6₁₋₂₀ peptide were given with monophosphoryl lipid A Sigma adjuvant, system (Sigma) in 200 μl according to the manufacturer's instructions. Proteins were administered 3 times at 2 weekly intervals except that in the experiment shown in FIG. 1B (see figure legend).

### 1.1.2 Pulmonary TB Challenge

Four to 5 weeks after the last immunisation with rec85A or ESAT-6₁₋₂₀ peptide or 10-11 weeks after BCG, mice were challenged i.n. with Mtb (Erdman strain, kindly provided by Dr Amy Yang, CBER/FDA). Mice were anaesthetised with isofluorane and 20 μl of Mtb suspension was pippetted into each nostril. Deposition in the lungs was measured 24 h after Mtb challenge and was ˜200 CFU per lung. Mice were sacrificed at 4-6 weeks after Mtb challenge. For time course studies mice were sacrificed at day 7, day 14, day 21 and day 28. Spleens and lungs were homogenized and the bacterial load was determined by plating 10-fold serial dilutions of tissue homogenates on Middlebrook 7H11 agar plates (E & O Laboratories Ltd, Bonnybridge, UK). Colonies were counted after 3-4 weeks of incubation at 37° C. in 5% CO₂.

### 1.2 Results

1.2.1 Almost Simultaneous Immunisation with BCG or Subunit Vaccines is Highly Effective

FIG. 1A shows that almost simultaneous i.n. and s.c. immunisation (i.e. approximately one hour between immunisations) is highly effective in protecting mice against challenge with pathogenic Mtb. In this experiment BCG given i.n. or s.c. reduced the mycobacterial load after challenge by ˜one log, while almost simultaneous i.n. and s.c. immunisation provided a further log reduction in load. The difference between single and almost simultaneous immunisation is statistically highly significant.

It was important to show that the additive effects of almost simultaneous immunisation are not due to the unique properties of BCG, which is known to trigger several pattern recognition receptors and to have tissue remodelling properties. We therefore examined the ability of a booster vaccine, recombinant mycobacterial antigen 85A protein (rec85A), to improve on protection induced by s.c. administered BCG, when given as an i.n. vaccine at almost the same time. FIG. 1B shows that rec85A i.n and BCG s.c. administered almost simultaneously, provide significantly increased protection compared to BCG alone. Almost simultaneous administration of BCG s.c. and rec85A s.c. does not improve protection over that delivered by BCG alone, indicating that it is essential to target both local lung and systemic immunity to achieve the additive protective effect of almost simultaneous immunisation.

### 1.2.2 The Mechanism of Almost Simultaneous Immunisation is not Priming and Boosting

A prime boost effect may underlie the improved protection seen when BCG s.c./BCG i.n. or BCG s.c./rec85A i.n. is administered almost simultaneously. However, the experiment shown in FIG. 1C confirms this not to be the case. Mice were immunised i.n., s.c. or almost simultaneously with two non-cross-reacting antigens, rec85A and a 20 mer synthetic peptide of the early secreted antigen ESAT-6. The combination of ESAT-6 s.c/rec85A i.n. gives significantly increased protection compared to either alone, confirming that almost simultaneous immunisation does not work through a prime boost mechanism.

1.2.3 Intranasal Vaccines that are Additive with Parenteral Vaccines Induce Early Protective Lung Immunity

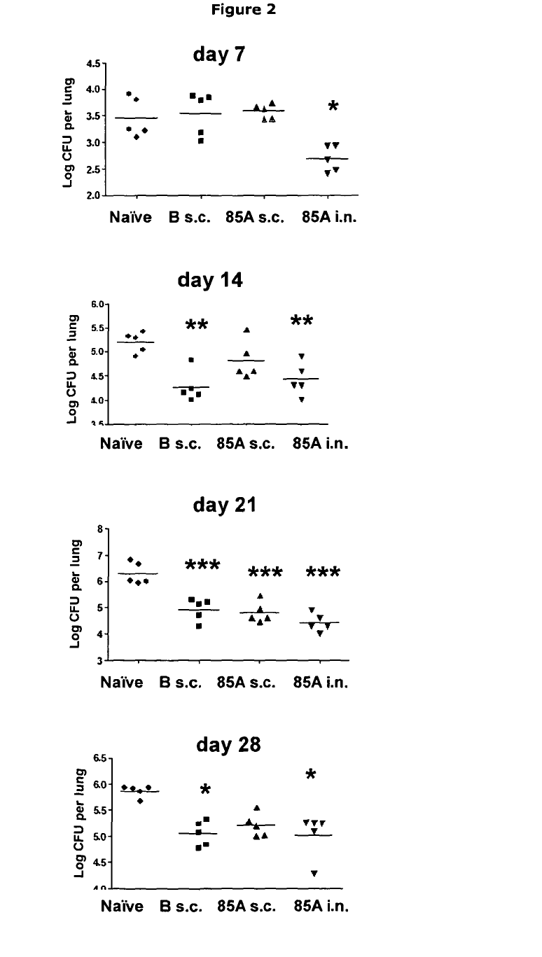

We have already shown that another model vaccine, the recombinant adenovirus Ad85A, given i.n. induces protective immunity able to inhibit early growth of Mtb after infection and we have postulated that this is why Ad85A provides additive protection when used as a booster after BCG (Ronan et al. 2009. PLoS One 4:e8235). FIG. 2 shows that the i.n. vaccine used here, rec85A protein with cholera toxin as adjuvant, also generates lung immunity able to inhibit mycobacterial growth in the first few days after infection. We conclude that i.n. administration of rec85A is additive with parenteral BCG because while BCG-immune animals can only inhibit growth in the late phase of Mtb infection, rec85A i.n. induces an immune response capable of inhibiting mycobacterial growth early after infection.

### Example 2

### 2.1 Materials and Methods

### 2.1.1 Animals and Immunisation

All experiments were performed with 6-8 week old female C57BL/6 mice (Harlan Orlac, Blackthorn, UK), were approved by the animal use ethical committee of Oxford University and fully complied with the relevant Home Office guidelines. Mice were immunized with 2×10⁵ colony forming units (cfu) BCG (SSI, Copenhagen, Denmark) s.c. on the flank in 200 μl PBS. For i.n. immunization, mice were anesthetized with isoflurane and 2×10⁵ cfu BCG in 40 μl PBS was administered with a pipette divided between the two nostrils. For SIM with BCG 1×10⁵ cfu were administered s.c. and 1×10⁵ cfu i.n. Mice were also immunized with 4 μg rec antigen 85A protein (85A), prepared as described previously (Franken, K. L., et al. Protein Expr Purif. 18, 95-99. (2000)), 20 μg of a synthetic peptide encoding the first 20 amino acids of 6 kDa early secreted antigenic target (E6) (Peptide Protein Research Ltd, Fareham, UK), 4 μg rec antigen TB10.4 (Proteix, Prague, Czech Republic) or 4 μg of the enduring hypoxia response protein Rv1284. Rv1284 was sub-cloned into the expression vector pET104-DEST42 (Invitrogen, Paisley, UK) from a complete Gateway Clone set from M. tuberculosis obtained through NIAID's Pathogen Functional Genomics Resource Center, managed and funded by Division of Microbiology and Infectious Diseases, NIAI, NIH, DHHS and operated by the J. Craig Venter Institute. After expression in BL21(DE3) E. coli cells (Invitrogen) as a His-tagged protein it was purified using His SpinTrap columns (GE Healthcare, Chalfont St Giles, UK). Subcutaneous (s.c.) immunization was performed by injecting each of the antigens, half subcutaneously and half intra-muscularly in 200 μl monophosphoryl lipid A Sigma adjuvant system (Sigma, Poole, UK) prepared according to the manufacturer's instructions. For i.n. protein immunization mice were anesthetized with isoflurane and the same doses of the antigens mixed with 2 μg of cholera toxin (Sigma) were pipetted into the nostrils in a total volume of 40 μl. Proteins were administered 3 times at 2 weekly intervals as indicated in the figure legends. Mice were also immunized once at the same time with BCG s.c. and either 4 μg 85A, 20 μg E6 or 4 μg TB10.4. The subunit vaccine was administered either s.c. with MPL or i.n. with CT as described above.

### 2.1.2 Isolation of Lymphocytes Lungs and Spleen

Lungs were perfused with PBS, cut into small pieces and digested with 0.7 mg/ml collagenase type I (Sigma) and 30 μg/ml DNase I (Sigma) for 45 ms at 37° C. Lung fragments were then crushed through a cell strainer using a 5 ml syringe plunger, washed, layered over Lympholyte (Cederlane, Ontario, Canada) and centrifuged at 1000×g for 25 ms. Interface cells were collected and washed. Spleens were passed through a cell strainer using a 5 ml syringe plunger, red blood cells were lysed using RBC lysis buffer (Qiagen, Crawley, UK) and the cells were washed.

### 2.1.3 Flow Cytometry

Cells were cultured in Hepes buffered RPMI supplemented with 10% heat-inactivated FCS, L-glutamine, penicillin and streptomycin for 6 hours. Cells were stimulated with PPD at 10 μg/ml (SSI) for 12 hours, or a pool of 66 15 mer peptides overlapping by 10 amino acids and covering the entire sequence of 85A or E6 20 mer (Peptide Protein Research Ltd) for 6 hours. Each peptide was at a final concentration of 2 μg/ml during the stimulation. After 6 (for PPD) and 2 (for the peptide pool) hours at 37° C., Golgi Plug (BD Biosciences, Oxford, UK) was added according to the manufacturer's instruction before intracellular cytokine staining.

Cells were washed and incubated with CD16/CD32 monoclonal antibody to block Fc binding. Subsequently the cells were stained for CD4 (RM4-5), CD8 (53-6.7) (BD Bioscience, Oxford, UK) IFNγXMG1.2 IL-2 (JES6-5H4) and TNF (MP6-XT22) (eBioscience, Hatfield, UK) using the BD Cytofix/Cytoperm kit according to the manufacturer's instructions. Cells were fixed with PBS+1% paraformaldehyde, run on a LSRII (BD Biosciences) and analyzed using FlowJo software (Tree Star Inc, Ashland, Oreg., USA).

2.1.4 Infection with Mtb and Determination of Mycobacterial Load

Five to 7 mice were anesthetized with isoflurane and infected i.n. with Mtb (Erdman strain, kindly provided by Dr. Amy Yang, CBER/FDA) in 40 μl PBS. Lung cfu were enumerated 24 hours after challenge to determine the number of organisms deposited, which was of the order of ˜200 cfu. Mice were sacrificed at indicated times, the lungs were homogenized and the lung mycobacterial load determined by plating 10-fold serial dilutions of tissue homogenates on Middlebrook 7H11 agar plates (E&O Laboratories Ltd, Bonnybridge, UK). Colonies were counted after 3-4 weeks of incubation at 37° C. in 5% CO₂.

### 2.1.5 Statistical Analysis

All results are representative of at least 2 independent experiments with similar results. Data were analyzed using one-way ANOVA followed by Tukey's multiple comparison test.

### 2.2 Results

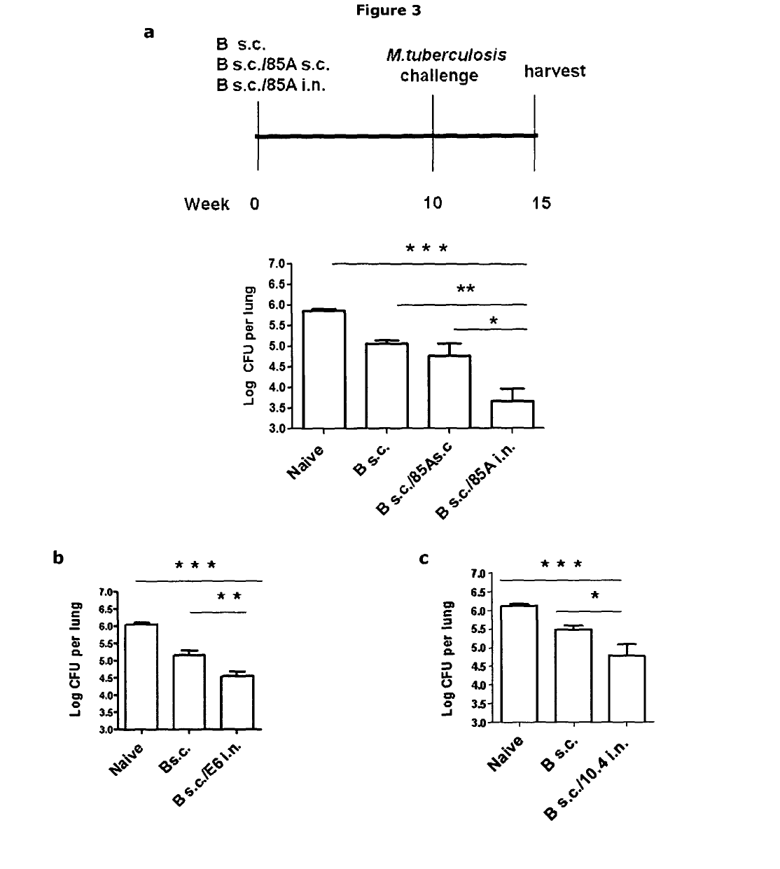

We first tested mice simultaneously with BCG subcutaneously (s.c.) and recombinant Mtb antigen 85A protein (85A) i.n. with cholera toxin (CT) as a mucosal adjuvant. SIM animals were compared to naive or BCG s.c. controls or mice given both BCG and 85A s.c. with monophosphoryl lipid A (MPL) as adjuvant. As expected, BCG s.c. suppressed Mtb growth by 0.8 log₁₀ compared to naive animals. However, SIM with BCG s.c./85A i.n., targeting both pulmonary and systemic immunity, provided strikingly increased protection, reducing Mtb colony forming units (cfu) by an additional 1.3 log₁₀ compared to the protection afforded by BCG s.c. alone. BCG s.c./85A s.c., targeting only systemic immunity, did not increase protection over BCG alone (FIG. 3a). To confirm the generality of this effect we carried out SIM with three other Mtb antigens, a synthetic peptide encoding the first 20 amino acids from the 6 kDa early secretory antigenic target ESAT6 (E6), rec protein TB10.4 and rec hypoxia induced protein Rv1284. Both BCG s.c./E6 i.n. or BCG s.c./TB 10.4 i.n. induced significant additional decreases in pulmonary Mtb load (by 0.6 log₁₀) compared to BCG alone (FIGS. 3b and c), while BCG s.c./E6 s.c. and BCG s.c./TB 10.4 s.c. did not (data not shown). Immunization s.c. or i.n. with Rv1284 did not generate protection in C57BL/6 mice assayed 5 weeks after Mtb infection (data not shown).

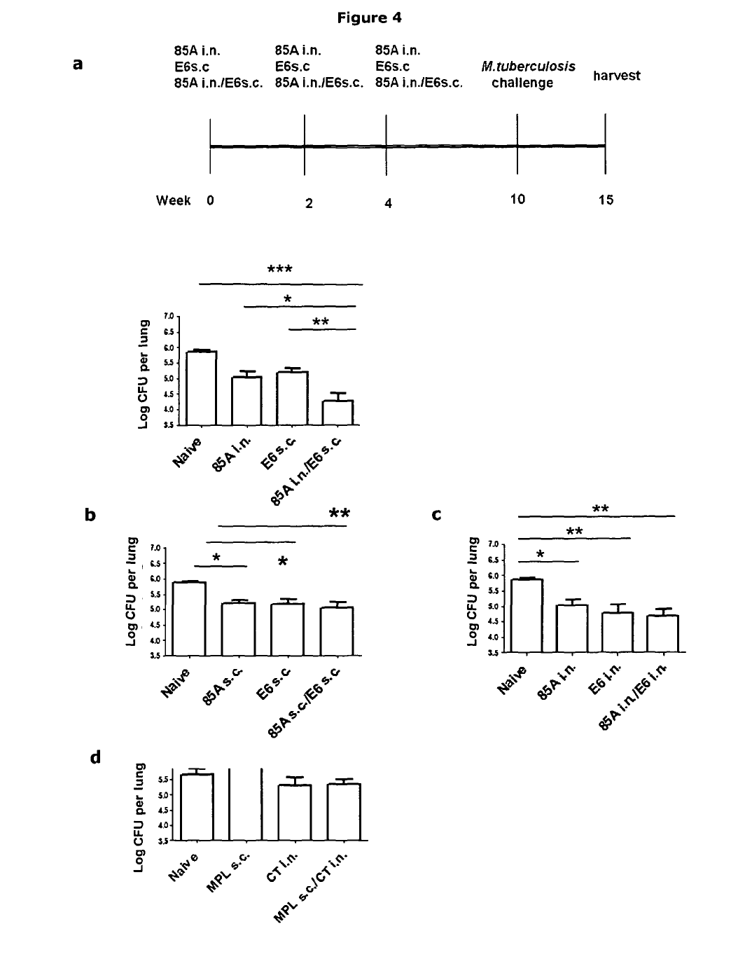

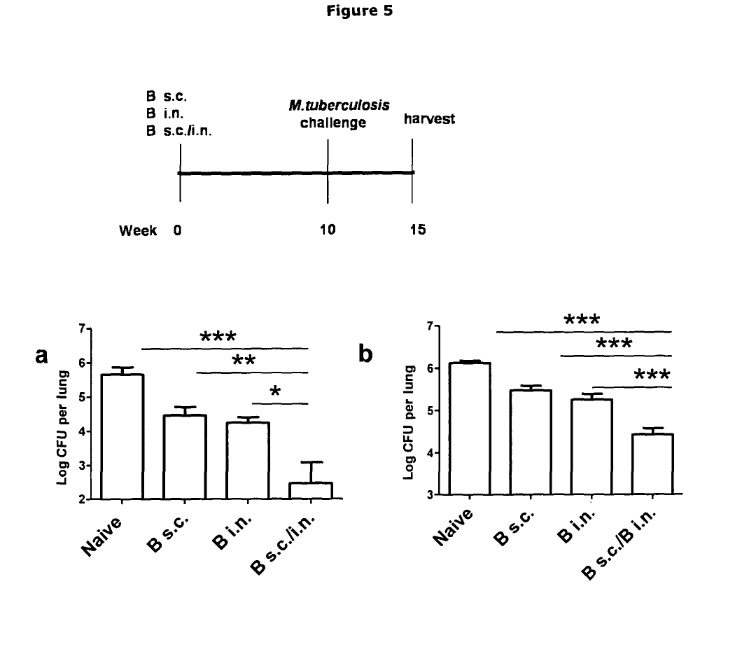

Since SIM with BCG s.c./E6 i.n. provided additional protection over BCG s.c. alone and BCG does not contain E6, this experiment suggested that the additional protection of SIM does not require priming and boosting. To establish this definitively, we used two non cross-reactive antigens, 85A and E6. Since adjuvanted rec protein vaccines are generally administered repeatedly (Skeiky. Y. A., et al. Vaccine 28, 1084-1093 (2010)), we gave the two subunit vaccines together or separately with appropriate adjuvants by the parenteral or pulmonary routes three times at two weekly intervals. The mice were challenged with Mtb 6 weeks after the last immunization (FIG. 4a). SIM with 85A and E6, targeting pulmonary and systemic immunity, decreased the mycobacterial load by 1.6 log₁₀ compared to naive mice. Immunization with one antigen by either the pulmonary or parenteral route alone had a lesser effect (FIG. 4a) and when both antigens were given together either s.c. or i.n., there was no significant increase compared to one subunit alone (FIG. 4b and c). We also immunized mice 3 times with CT i.n., MPL s.c. or both simultaneously and challenged them with Mtb. The mycobacterial burden in these controls did not differ from that found in naive animals (FIG. 4d). Finally, since BCG is the gold standard vaccine for animal experiments and the only available human tuberculosis vaccine, we tested SIM with BCG s.c./BCG i.n. This combination was highly effective, reducing the mycobacterial load in two separate experiments by an additional 1.1 and 2.0 log₁₀ respectively compared to BCG given by either route alone (1.7 and 3.1 log₁₀ compared to naïve mice) (FIG. 5a and b).

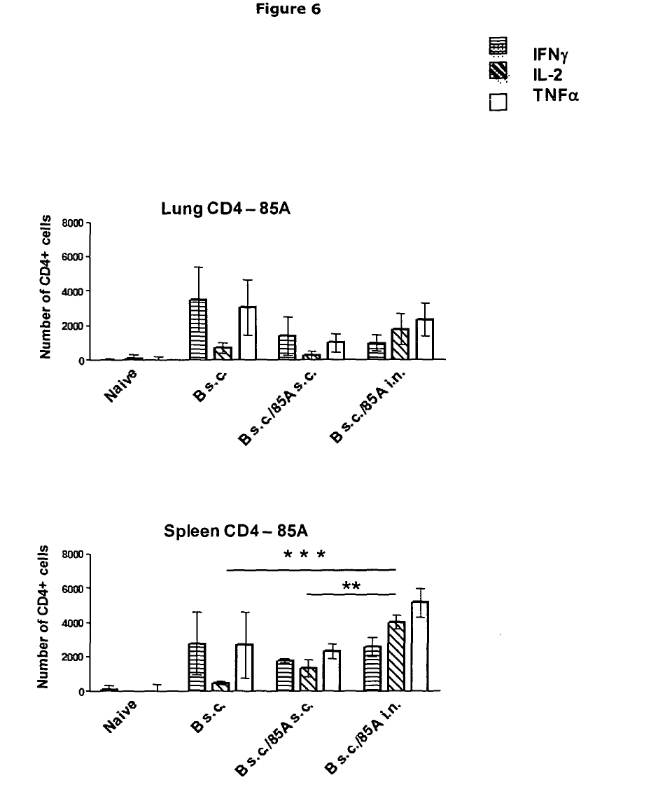

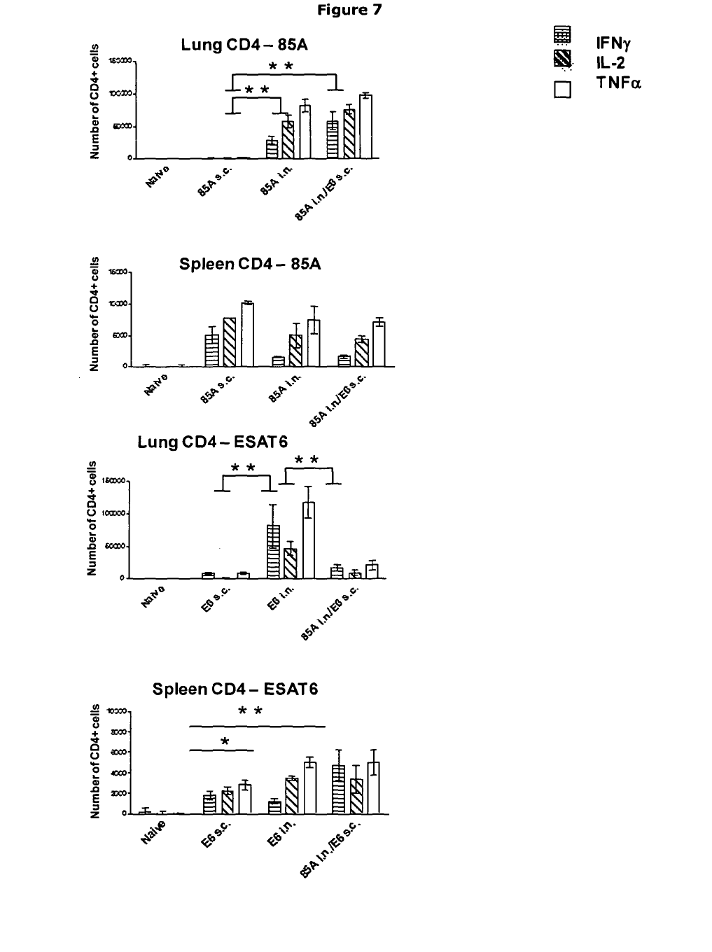

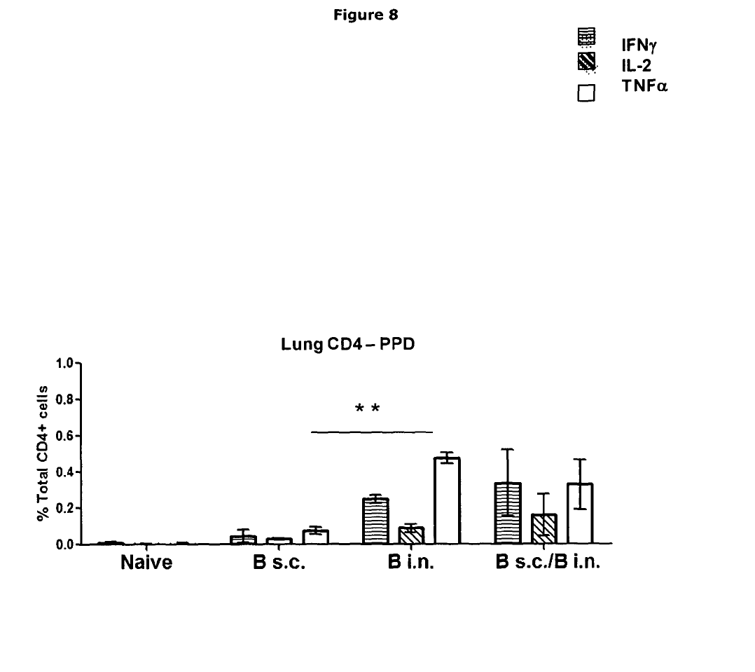

To investigate the mechanisms underlying the efficacy of SIM, we assayed antigen specific responses in the lungs and spleen at the time of Mtb challenge by intra-cytoplasmic cytokine staining (ICS) and flow cytometry. Ten weeks after SIM with BCG and 85A (for description of experiment see FIG. 3a), there were few antigen 85A specific CD4 cells in the lungs (<5,000). In the spleen, responses were strongest in mice given BCG s.c./85A i.n. rather than those given BCG s.c./85A s.c. There was no evidence at this time point for a booster effect of SIM in mice that received BCG and 85A (FIG. 6). In contrast, six weeks after 3 immunizations with 85A or E6 vaccines i.n. (FIG. 4a), powerful lung responses were seen (>50,000 antigen specific CD4 cells) while s.c. immunization induced very low lung responses. In all groups there were no more than 10,000 antigen specific cells in the spleen, but responses following i.n. immunization were comparable to those after s.c. immunization, confirming the efficacy of mucosal immunization. When two different antigens were given, the responses to each were largely unaffected (FIG. 7). Ten weeks post immunization with BCG i.n. or BCG s.c./BCG i.n. (FIG. 5) 0.2-0.5% of lung CD4 cells produce cytokines in response to PPD while after BCG s.c.<0.1% do so (FIG. 8). All splenic responses were too low to measure reliably by ICS (data not shown).

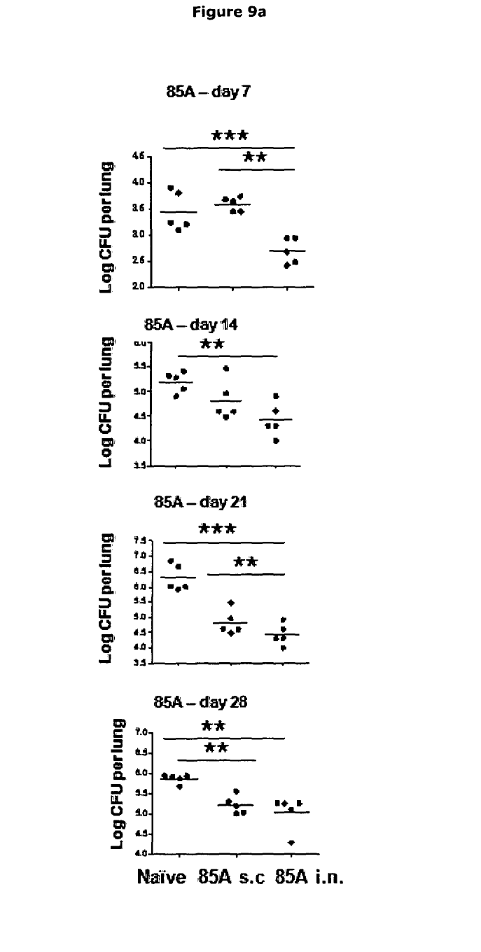

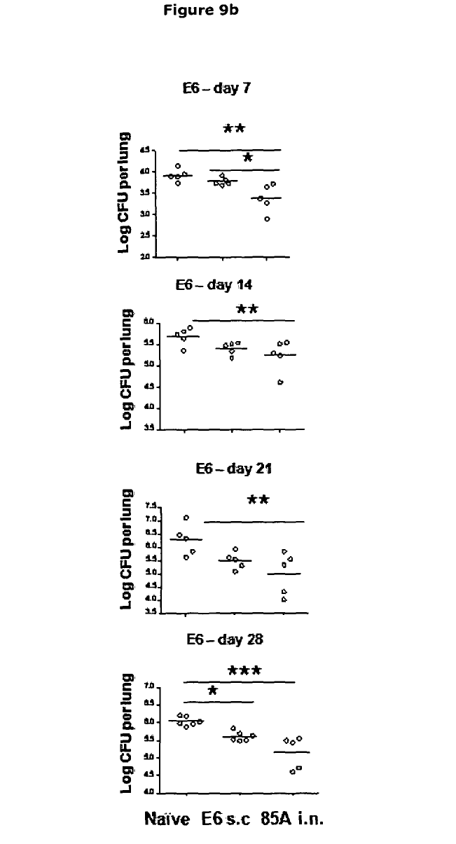

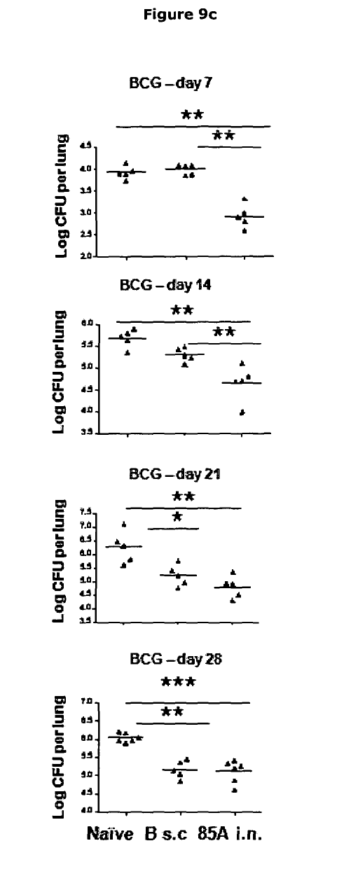

We also analyzed numbers of single or multiple cytokine producing cells in lungs and spleens (data not shown) but we could not discern any correlation between either the numbers or quality of antigen specific cells and protective immunity. However, after i.n. immunization antigen specific cells are always present in the lungs, so we hypothesized that these might be inhibiting Mtb growth immediately after infection. We therefore examined the kinetics of Mtb growth after immunization and challenge (FIG. 9). Pulmonary vaccines that provided addition protection with parenteral vaccines had inhibited Mtb growth by 7 days post challenge, whereas effective parenteral vaccines, including BCG, only inhibited growth 14 days or later after challenge (FIG. 9).

### 2.3 Conclusions

SIM with BCG s.c./BCG i.n. is highly effective. However, BCG is known to affect innate immunity and even tissue remodelling and we do not yet know whether these contribute to protection, nor do we yet know the duration of increased protection. Nevertheless the extraordinary efficacy of SIM with BCG s.c./BCG i.n. (FIG. 5) sets a new gold standard against which to measure Mtb vaccine efficacy. Another SIM regime, BCG s.c./85A i.n., is highly effective (FIG. 3a) but clearly there are many possibilities for improvement of SIM with subunits, such as the use of multiple antigens (including latency antigens) as fusion genes, or recombinant mycobacteria over-expressing immunogenic antigens. Adjuvants and vectors for pulmonary delivery need to be further developed. SIM has several potential advantages: first it is highly effective; second it only requires immunization procedures that might be performed at a single clinical visit; third it is compatible with further boosting with subunit vaccines or the employment of recombinant mycobacteria. Some of these are as effective as BCG in inducing protective immunity but may be less pathogenic, an important property in HIV+ immuno-compromised individuals. The safety and long-term efficacy of pulmonary vaccines remain to be thoroughly investigated. However, cheap and efficient delivery methods for pulmonary vaccination have been developed, respiratory immunization against measles has been shown to be safe and highly efficient, and BCG itself has been safely administered to the lungs of humans. SIM is a novel strategy, with the potential to enhance the efficacy of existing promising subunit parenteral vaccines, and requires further investigation and development.

1.-25. (canceled)

26. A method for immunising a subject against Mycobacterium tuberculosis or Mycobacterium bovis, the method comprising (a) parenterally administering to the subject an immunologically effective amount of a first antigen from the Mycobacterium or a first polynucleotide encoding the first antigen and (b) administering to the lung of the subject an immunologically effective amount of a second antigen from the Mycobacterium or a second polynucleotide encoding the second antigen, wherein steps (a) and (b) are carried out less than four weeks apart.

27. A method according to claim 26, wherein steps (a) and (b) are carried out less than three weeks apart, less than two weeks apart, or less than one week apart.

28. A method according to claim 26, wherein steps (a) and (b) are carried out less than 48 hours apart, less than 36 hours apart, or less than 24 hours apart.

29. A method according to claim 26, wherein step (a) is carried out before step (b).

30. A method according to claim 26, wherein step (b) is carried out before step (a).

31. A method according to claim 26, wherein steps (a) and (b) are carried out simultaneously.

32. A method according to claim 26, wherein the first and second antigens are the same.

33. A method according to claim 26, wherein the first and second antigens are different.

34. A method according to claim 26, wherein at least one of the first and second antigen is the 85A, 85B or 85C antigen from Mycobacterium tuberculosis or Mycobacterium bovis or a variant thereof, or wherein at least one of the first and second antigen comprises SEQ ID NO: 2 or a variant thereof.

35. A method according to claim 26, wherein at least one of the first and second antigen is the TB 10.4 antigen from Mycobacterium tuberculosis or Mycobacterium bovis or a variant thereof.

36. A method according to claim 26, wherein at least one of the first and second antigen is the enduring hypoxia response protein Rv1284 from Mycobacterium tuberculosis or Mycobacterium bovis or a variant thereof.

37. A method according to claim 26, wherein at least one of the first and second antigen is administered as part of an attenuated mycobacterium or a recombinant attenuated mycobacterium.

38. A method according to claim 26, wherein (a) at least one of the first and second antigen is administered as part of an attenuated version or a recombinant attenuated version of the Mycobacterium tuberculosis or Mycobacterium bovis; (b) at least one of the first and second antigen is administered as part of Bacillus Calmette-Guérin (BCG) or recombinant BCG; (c) at least one of the first and second antigen is administered as part of live BCG; or (d) at least one of the first and second antigen is administered as part of recombinant BCG which has been modified to make it more immunogenic.

39. A method according to claim 38, wherein the recombinant attenuated Mycobacterium tuberculosis is non-pathogenic and immunogenic.

40. A method according to claim 26, wherein at least one of the first antigen and the second antigen is the 6 kDa early secreted antigenic target (ESAT-6) from Mycobacterium tuberculosis or a variant thereof, or wherein at least one of the first and the second antigen comprises SEQ ID NO: 4 or a variant thereof.

41. A method according to claim 26, wherein the first and second antigens do not cross-react.

42. A method according to claim 26, wherein step (a) induces a systemic mononuclear cell response to the antigen and step (b) induces a pulmonary mononuclear cell response to the antigen.

43. A method according to claim 26, wherein the subject is a human or a cow.

44. A kit for immunising a subject against Mycobacterium tuberculosis or Mycobacterium bovis, the kit comprising (a) an immunologically effective amount of a first antigen from the Mycobacterium or a first polynucleotide encoding the first antigen which is suitable for parenteral administration to the subject and (b) an immunologically effective amount of a second antigen from the Mycobacterium or a second polynucleotide encoding the second antigen which is suitable for administration to the lung of the subject.


SEQUENCE LISTING (4 sequences)

SEQ ID NO: 1 - DNA, 1299 bp, Mycobacterium bovis
  CDS (91)..(1107)
actgccgggcccagcgcctgcagtctgacctaattcaggatgcgcccaaacatgcatggatgcgttgagatgaggatgagggaagcaagaatgcagcttgttgacagggttcgtggcgccgtcacgggtatgtcgcgtcgactcgtggtcggggccgtcggcgcggccctagtgtcgggtctggtcggcgccgtcggtggcacggcgaccgcgggggcattttcccggccgggcttgccggtggagtacctgcaggtgccgtcgccgtcgatgggccgtgacatcaaggtccaattccaaagtggtggtgccaactcgcccgccctgtacctgctcgacggcctgcgcgcgcaggacgacttcagcggctgggacatcaacaccccggcgttcgagtggtacgaccagtcgggcctgtcggtggtcatgccggtgggtggccagtcaagcttctactccgactggtaccagcccgcctgcggcaaggccggttgccagacttacaagtgggagaccttcctgaccagcgagctgccggggtggctgcaggccaacaggcacgtcaagcccaccggaagcgccgtcgtcggtctttcgatggctgcttcttcggcgctgacgctggcgatctatcacccccagcagttcgtctacgcgggagcgatgtcgggcctgttggacccctcccaggcgatgggtcccaccctgatcggcctggcgatgggtgacgctggcggctacaaggcctccgacatgtggggcccgaaggaggacccggcgtggcagcgcaacgacccgctgttgaacgtcgggaagctgatcgccaacaacacccgcgtctgggtgtactgcggcaacggcaagccgtcggatctgggtggcaacaacctgccggccaagttcctcgagggcttcgtgcggaccagcaacatcaagttccaagacgcctacaacgccggtggcggccacaacggcgtgttcgacttcccggacagcggtacgcacagctgggagtactggggggcgcagctcaacgctatgaagcccgacctgcaacgggcactgggtgccacgcccaacaccgggcccgcgccccagggcgcctagctccgaacagacacaacatctagcggcggtgacccttgtggtcgccgccgtagatgtttcctaaatcccgtccctagctcccgccgcgggccgtgtggttagctacctgacgggctaggggttggccggggcggttgacgccgggtgcacacagcctacacgaacggaaggtggacacatgaagggtcggtc

SEQ ID NO: 2 - protein, 338 aa, Mycobacterium bovis
MQLVDRVRGAVTGMSRRLVVGAVGAALVSGLVGAVGGTATAGAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGA

SEQ ID NO: 3 - DNA, 288 bp, Mycobacterium tuberculosis
  CDS (1)..(288)
atgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcatag

SEQ ID NO: 4 - protein, 95 aa, Mycobacterium tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFA